SEQUENCE LISTING (6 sequences)

SEQ ID NO: 1 - DNA, 34 bp, Canis familiaris
aatccatgggcaacgccgccgccaagaagggcag

SEQ ID NO: 2 - DNA, 37 bp, Canis familiaris
gccgtcgacgaactcacaaaactccttgccacacttc

SEQ ID NO: 3 - DNA, 18 bp, Canis familiaris
aatacgactcactatagg

SEQ ID NO: 4 - DNA, 19 bp, Canis familiaris
gctagttattgctcagcgg

SEQ ID NO: 5 - protein, 350 aa, Canis familiaris
MGNAAAKKGSEQESVKEFLAKAKEDFLKKWENPAQNTAHLDQFERIKTLGTGSFGRVMLVKHKETGNHFAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFCEF

SEQ ID NO: 6 - DNA, 1053 bp, Canis familiaris
atgggcaacgccgccgccaagaagggcagcgagcaggagagcgtgaaagagttcttagccaaagccaaagaagactttcttaaaaaatgggaaaatcctgctcagaatacagcccatttggatcagtttgaacgaatcaagaccctcggcacgggctcctttgggcgggtgatgctggtgaagcacaaggagaccgggaaccactttgccatgaagatcctcgacaaacagaaggtcgtgaagctgaaacagattgagcataccctgaacgaaaagcgcatcctgcaagcggtcaacttcccgttcctcgtcaaactcgagttctccttcaaggacaactcgaatttatacatggtcatggagtatgtgcctggcggggagatgttctcgcacctgcggcggatcggaaggttcagtgaaccgcatgcccgcttctatgcggcccagatcgtcctgaccttcgagtacctgcactcacttgacctcatctaccgggacctgaagccggagaacctcctcattgaccagcagggctacattcaggtgacagacttcggttttgccaagcgtgtaaaaggccgcacctggaccttatgcgggacccccgagtacctggccccggaaatcatcctgagcaaaggctacaacaaggctgtggactggtgggccctaggggttctcatctacgagatggccgctggctacccacccttctttgctgaccagcccatccagatctatgagaagattgtctctgggaaggtgcggttcccgtcccacttcagctctgacctgaaggatctgctacggaacctgttgcaggtggacctcaccaaacgcttcgggaacctcaagaatggggtgaacgatatcaagaaccacaagtggtttgccacaactgactggatcgccatctatcagaggaaggtggaagctcccttcataccaaagtttaaaggccctggggacacgagtaattttgacgactatgaggaggaagagatccgagtctccatcaatgagaagtgtggcaaggagttttgtgagttctag

(19) United States
(12) Patent Application Publication
(10) Pub. No.: US 2020/0141939 A1
(43) Pub. Date: May 7, 2020

(54) APPARATUS AND METHOD FOR DETECTING CANINE CANCER

(71) Applicant: Dongha Bang, Seoul (KR)

(72) Inventor: Dongha Bang, Seoul (KR)

(21) Appl. No.: 16/337,114

(22) PCT Filed: Oct. 10, 2017
(86) PCT No.: PCT/IB2017/056236

§ 371 Date: Mar. 27, 2019

Related U.S. Application Data
(60) Provisional application No. 62/405,996, filed on Oct. 10, 2016.

(51) Int. Cl. G01N 33/574; C12N 9/12; G01N 33/543; G01N 21/75; G01N 33/68

(52) U.S. Cl. CPC G01N 33/57488; C12N 9/12; C12Y 207/11011; G01N 21/75; G01N 33/6854; G01N 33/54386

(57) ABSTRACT

It has been found that canine ECPKA protein secrets in a high level and an autoantibody against the canine ECPKA protein is formed in dogs with cancer. It is also found that human ECPKA does not selectively bind to a canine ECPKA autoantibody and cannot serve as a biomarker. In addition, canine ECPKA autoantibody detection can be used as a meaningful diagnosis tool for cancer in dogs only when quantitative measurement of such antibodies is adapted. When the measurement of canine ECPKA autoantibody is not conclusive, measuring CRP can provide supplemental data that can be used to improve the predictability of the canine ECPKA autoantibody measurement.

### CROSS-REFERENCE TO RELATED APPLICATIONS

This application claims the benefit under 35 U.S.C. Section 371, of PCT International Application No. PCT/IB2017/056236, filed on Oct. 10, 2017, which claimed priority to U.S. Application 62/405,996, filed on Oct. 10, 2016, the disclosures of which are hereby incorporated by reference herein in their entirety.

### SEQUENCE LISTING

A sequence listing has been submitted with this invention and is incorporated by reference herein, in its entirety.

### FIELD OF THE INVENTION

The present invention relates to apparatus and methods for detecting cancer of a dog using canine PKA Cα protein as an antigen. The detection method and apparatus utilize quantitative detection of antibodies that can be bound to a canine PKA Cα protein or its subunit with a therapeutically meaningful selectivity and specificity.

### BACKGROUND

Human and dogs share many similarities genetically and immunologically but at the same time, exhibit many different immune responses and biological responses as well. For example, humans are susceptible to AIDS virus but dogs are resistant to the virus. Also, dogs are susceptible to canine distemper but humans are not.

There are many other examples where a potential bio-marker useful for human but not to dogs. For example, the S phase-specific protein thymidine kinase 1 (TK1) has been known to be useful biomarker for human cancer as described in H. von Euler and S. Eriksson, Vet Comp Oncol. 2011 March, 9(1):1-15. doi: 10.1111/j.1476-5829.2010.00238.x. Epub 2010 Aug. 19. The expression of TK1 is tightly correlated to the fraction of S phase cell and the level of proliferation. In normal cells, TK1 activity is present only during the late G1 and early S phase but in many tumor cells, TK1 activity is higher and remains throughout the S and G2 phases. Excessive and uncontrolled cell proliferation is one of hallmarks of cancer. Thus, the level of TK1 activity was examined to determine its correlation with cancer and has proven useful for diagnosis of and monitoring tumors such as solid tumor including breast cancer. Moreover, a major advantage in TK1 is that several monoclonal and polyclonal antibodies can be bound to TK1. The most specific and sensitive TK1 antibodies are produced against a 31-amino acid peptide representing the C-terminus of TK1. The protein sequence homology in TK1 is high between humans and dogs. H. von Euler and S. Eriksson. The primary amino acid sequences of canine and human TK1 are highly homologous from the N-terminal and for about 200 amino acids but the C-terminal regions differ.

In spite of the high homology in the protein sequence between human and canine, the TK1 values in dogs having four different solid tumors including breast cancer were found to be within a normal range. Nakamura N, Momoi Y, Watari T, Yoshino T, Tsujimoto H and Hasegawa A., Plasma thymidinekinase activity in dogs with lymphoma and leukemia, the Journal of Veterinary Medical Science 1997; 59: 957-960. Also, only three of 50 tested dogs with a solid cancer showed an increased TK1 activities. Monitoring therapy in canine malignant lymphoma and leukemia with serum thymidine kinase 1 activity—evaluation of a new, fully automated non-radiometric assay. International Journal of Oncology 2008; 34: 505-510. Canine TK1 is not a useful biomarker for detecting cancer for dogs unlike human TK1. Moreover, the most sensitive and selective antibodies directed against human TK1 do not recognize canine TK1. H. von Euler and S. Eriksson. Similarly, antibodies directed canine TK1 at similar region cannot likely be recognized by human TK1 protein.

Cyclic AMP (cAMP)-dependent protein kinase A (PKA), a serine/threonine protein kinase mediating cAMP action in mammalian cells, is involved in controlling various biological processes such as cell proliferation, differentiation, metabolism and apoptosis. Deregulation of PKA has been linked with the initiation and progression of cancer, and indeed its overexpression is frequently observed in different types of human cancer.

Hence, PKA has been suggested as a potential molecular target for a diagnostic biomarker in human cancer. Inactive PKA holoenzyme is a tetramer composed of two catalytic and two regulatory subunits. Upon binding with cAMP on regulatory subunits, the inactive PKA tetramer is dissociated into one dimer of regulatory subunits and two monomers of active catalytic subunits, which then phosphorylate various target proteins in both nucleus and cytoplasm. Four isoforms of regulatory subunit, RIα, RIβ, RIIα and RIIβ, have been identified through biochemical studies and outcomes of PKA signaling activation has been shown to depend on types of regulatory subunit isoforms in cells. The expression of RII isoforms is preferentially observed in normal tissues and inhibits the growth of cells, whereas the expression of RI isoforms (RIα/PKA-I) stimulates cell proliferation.

In particular, overexpression of the RIα/PKA-I is highly correlated with cancer progression, multidrug resistance and various types of cancer patients with a poor prognosis. Interestingly, recent studies show that PKA is expressed mostly intracellularly in normal cells, whereas an extracellular form of the PKA (ECPKA) is secreted from numerous types of cancer cell lines including prostate, bladder, breast, colon carcinoma and lung adenocarcinoma. In addition, ECPKA was highly detected in sera from human patients with various types of cancer but not in sera from healthy volunteers. Furthermore, it was also shown that the serum level of ECPKA decreases after surgical resection of tumors in human melanoma patients. See U.S. Pat. No. 7,838,305B2, which report compositions and methods for the detection of anti-ECPKA autoantibodies using human ECPKA as an antigen. These observations raise the possibility that human ECPKA and its autoantibodies in serum could be a valuable diagnostic for human cancer.

Canine ECPKA has different amino acid sequence with human ECPKA. The amino acid sequences are shown in FIG. 1. The different sequences are spread throughout the chain including C-terminus. It is not known whether canine ECPKA present in dogs with cancer and, in particular, whether the level of the canine ECPKA present in dogs can be correlated with the presence of cancer. Moreover, it is not known whether dogs have immune responses to canine ECPKA creating autoantibodies or even if so, whether such immune response strong enough to be useful for a biomarker to detect canine cancer. It is also not known whether human ECPKA can be used as an antigen for a diagnostic purpose to detect canine cancer. Typically, a level of antigen such as ECPKA in a plasma may decay over time and it may be difficult to adopt a quantitative analysis of an antigen for a diagnostic measurement. The detection of a level of antigen may not be a useful tool for diagnosis or detection with a certain probability because the retention time of the sample before subjecting the test would affect the result. However, a level of an autoantibody is much more free of such decay and can be useful as a biomarker especially when a quantitative control is required.

Through extensive research and studies over different breeds and ages of dogs with various types of cancer, it has been found that dogs show distinctive canine ECPKA and autoantibody activities, which can be used to detect the presence of cancer in a dog and there is no or very little correlation between human ECPKA and canine cancer detection. Due to the unique distinctiveness, a quantitative detection of the antibodies for canine ECPKA, rather than a simple qualitative detection, is desirable and a device enabling such quantitative detection is developed. In particular, the quantitative detection is developed to be adopted in digitized data process and a device assisting such digitized process is also developed. The quantitative detection device can be used for detecting other proteins or virus agents as described herein.

### SUMMARY OF INVENTION

One embodiment provides a method for determining presence of cancer in a dog by preparing a serum sample from the dog and detecting an amount of an antibody in the serum sample using a purified recombinant canine PKA Cα protein as an antigen wherein the presence of cancer in the dog is determined when the amount of the canine PKA Cα antibody is above a predetermined level. Depending on the detected amount of the canine PKA Cα antibody, the possibility of the cancer presence can be determined. The purified recombinant canine PKA Cα protein is synthesized by using a primer with a sequence SEQ ID NO. 1 (AAT CCA TGG GCA ACG CCG CCG CCA AGA AGG GCA G) and SEQ ID NO. 2 (GCC GTC GAC GAA CTC ACA AAA CTC CTT GCC ACA CTT C). The purified recombinant canine PKA Cα protein is prepared by using amplified cDNA fragments of canine PKA Cα and a bacterial expression vector with T7 promoter and terminator primers having sequences respective SEQ ID NO. 3 (AAT ACG ACT CAC TAT AGG) and SEQ ID NO. 4 (GCT AGT TAT TGC TCA GCG G). The resulting purified recombinant canine PKA Cα protein has an amino acid sequence comprising SEQ ID NO. 5 and a nucleotide sequence comprising SEQ ID NO. 6.

The predetermined level can be determined in consideration of various factors such as presence of other medical conditions such as liver disease or inflammatory conditions. For example, dogs with a liver disease may have exhibit a higher level of canine ECPKA autoantibodies. Thus, the method may have a preset instruction for dogs without a liver disease. The predetermined level may be 3.5 μg/ml, preferably 4 μg/ml, more preferably 4.5 μg/ml, even more preferably 5 μg/ml.

It is also found that a high level of the amount of CRP is determined in dogs with cancer. Accordingly, determining the amount of CRP can be used in conjunction with the ECKPA or ECPCKA autoantibody detection to determine the presence of cancer in a dog. Because CRP may presence much more predominantly in the serum than the ECPKA or ECPKA autoantibodies, a higher level of the CRP amount can be set to be a threshold level. Moreover, the CRP amount may be measured after further diluting the serum sample using a buffer solution. The dilution factor may be 100 times. The predetermined CRP level may be about 80 μg, preferably 100 μg or even more preferably 120 μg.

Another embodiment provides a method for determining presence of cancer in a dog preparing a serum sample from the dog detecting an amount of a canine PKA Cα antibody in the serum sample using a purified recombinant canine PKA Cα protein as an antigen and determining an amount of CRP in the serum sample, wherein the presence of cancer in the dog is determined when the amounts of the canine PKA Cα antibody and CRP are respectively above a predetermined antibody level and a predetermined CRP level and wherein the amount of CRP is measured with an assistance of a diluting buffer solution. The dilution buffer solution dilutes the serum sample by a factor of between about 50 and about 150.

Another embodiment provides a device for quantitatively detecting an antibody for canine PKA Cα protein a solid phase having an immobilized purified recombinant canine PKA Cα protein wherein the recombinant canine PKA Cα protein has an amino acid sequence comprising SEQ ID NO. 5, wherein the device is capable of detecting an amount of the antibody for canine PKA Cα protein using the recombinant canine PKA Cα protein. When the detected amount is above a predetermined level, cancer may presence in a high possibility. The device for quantitatively detecting an antibody for canine PKA Cα protein may include a ligand capable of binding canine lgG wherein the ligand including a portion that is active to either UV or visible light. The device may include a housing with a sample reception portion, detection window wherein the solid phase is placed within the housing and is capable of moving a sample received from the sample reception potion through a portion of the solid phase exposed by the detection window.

The device for quantitatively detecting an antibody for canine PKA Cα protein may be able to determine at least two levels of the amount of the detected antibody. The device may have a reference line which can be used as a reference quantitative line. The reference line shows a similar color result regardless of the sample and allows quantitative determination of the amount of the antibody detected. The device is preferably capable of determining multiple levels of the amount of the detected antibody and gaps between two adjacent levels of the multiple level is less than about 5 μg/ml or less, preferably 3 μg/ml or less, even more preferably 1 μg/ml or less.

The device for quantitatively detecting a canine ECPKA autoantibody may include one or more data input slots, which allows simultaneous collection of patient data while the result of the device is taken. For example, the result of the device can be read using a reader by taking a photo of the device and the test result can be read and converted to a digital data. The conversion process can convert the image of the solid phase shown in the detection window and also at the same time, convert the data shown in the data input slots into a digital data for process. Such converted data can be sent to a remote server through a communication device or module that may be incorporated in the test reader. The transmitted data can be compiled and organized for analyzing. The analyzed data can be sent back to the test performer via the test reader or other electronic means such as email, message, etc. The test reader may use a smart phone to take a photo and transmit the photo to the remote server.

Another embodiment provides am apparatus for detecting a protein or viral agent in a mammal may have a housing with an outer surface and an inner space, a control panel on the outer surface, a camera located in a way to be able to take a picture of a protein or viral detection sample in the inner space, a sample holder formed in the inner space wherein the sample can be placed on the sample holder, a light source capable of illuminating the inner space; a light dissipating device configured to dissipate light from the light source allowing indirect illumination on the sample, a communication module, wherein the light source and the light dissipating device are configured to indirect illumination of the light from the light source on the sample holder and the communication module is configured to send the picture taken by the camera to a remote server.

The sample holder is configured to hold a lateral flow kit or other bio assay kit. The sample holder allows to locate the sample within a range of the camera so that the camera can take a picture of the sample. The camera and the communication module may be incorporated into the housing. Alternatively, the camera and communication are part of a smart phone where the smart phone is placed on the housing to take a picture of the sample and send the picture to a remote server. The smart phone's flash light can be used the light source. The light dissipating device may be a semitransparent plate and is movable to be located directly beneath of the light source of the smart phone.

The apparatus detecting a protein or viral agent in a mammal may include a removable smart phone adopter having an camera opening wherein the light dissipating device is incorporated into the smart phone adopter in a way to cover the light source in the smart phone and the camera opening is aligned to the camera of the smart phone wherein the housing further comprising a receiving area for the removable smart phone adopter is placed wherein the receiving area has one or more opening for the camera and the light source. The smart phone adopter may be incorporated into the housing.

The light source may be capable of illuminating light with a predetermined wave length such as UV light or mono wave light. The communication module may use a short distance communication protocol such as WiFi, WiMx, Bluetooth, ZigBee, Z-Wave or other short distance communication protocol.

Another embodiment provides a method of detecting and monitoring an infectious disease in a mammal includes testing the infectious disease in a mammal using a kit having a viral detection agent, determining the result of testing using a kit reader with camera and communication capabilities, sending the test result obtained by the kit reader to a remote server; and alerting a predetermined entity by an electrical communication method is provided. The method may utilize a kit that provides a UV active result and the camera is capable of detecting the US active result.

The kit reader may include a housing having an outer surface and an inner space, a control panel on the outer surface, a sample holder formed in the inner space wherein the sample can be placed on the sample holder, a light source capable of illuminating the inner space; and a light dissipating device configured to dissipate light from the light source, wherein the camera located in a way to be able to take a picture of a protein or viral detection sample in the inner space, the light source and the light dissipating device are configured to indirect illumination of the light from the light source on the sample holder and the communication module is configured to send the picture taken by the camera to a remote server.

The test reader or kit reader may have GPS capability and is able to identify the location of the test.

Another embodiment provides a lateral flow kit for quantitatively detecting an antibody of canine ECPKA in blood of a dog, having a first solid phase having an immobilized purified recombinant canine PKA Cα protein wherein the recombinant canine PKA Cα protein has an amino acid sequence comprising SEQ ID NO. 005, a conjugate pad comprising a conjugated coloring agent with an optical density wherein the conjugated coloring agent is conjugated with a first binding protein, and a second solid phase comprising a second binding protein, wherein the conjugated coloring agent is configured to provide a first color intensity in the first solid phase and a second color intensity in the second solid phase and wherein the first color intensity quantitatively depends on the concentration of the antibody in the blood of the dog and the second color intensity is independent of the concentration of the antibody.

Because the concentration of the antibody is measured quantitatively using simple the lateral flow kit, the coloring agent may be applied using a pressurizing device and the optical density of the coloring agent may preferably be used in a high level. The optical density of the conjugated coloring agent may be between 5-30, preferably 8-25, more preferably 10-20.

The first color intensity provides the concentration of the antibody by comparing the first color intensity with a set of correlating data between the first color intensity and the concentration of the antibody. The concentration can be extrapolated using the set of the data, which can be expressed into a correlation equation, preferably a linear equation.

The first binding protein may be selected from a group of streptavidin, biotin, protein A, anti-canine IgG Rat, anti-canine IgG Rabbit, anti-canine IgG goat, anti-canine IgG sheep and a protein capable of binding to the antibody. The second binding protein is selected from a group of streptavidin, biotin, protein A, anti-rat IgG, anti-Rabbit IgG, anti-goat IgG, anti-sheep IgG and a protein capable of binding to IgG. Selection of the first and second binding proteins needs to be complimentary.

The coloring agent may be selected from a group of gold, latex, gfp, fitc, and UV active conjugating agent. Different sizes of gold nano particles may be used.

The lateral flow kit may further have a filter phase to filter blood cells, which allows directly applying a blood sample rather than serum.

Another embodiment provides a method of determining a concentration of an antibody of canine ECPKA in blood of a dog where the method includes steps of (a) preparing a test sample from the blood of the dog for a lateral flow kit comprising a test line; (b) applying the test sample to the lateral flow kit; (c) allowing the lateral flow kit to develop; (d) obtaining a digital information by taking a digital picture of the developed lateral flow kit including the test line; and (e) obtaining the concentration of the antibody wherein the concentration is determined by comparing the digital information of the test line with a set of data correlating a digital value obtained from the digital picture with a concentration of the antibody.

The lateral flow kit used in this embodiment may be the lateral flow kit described herein. The method may also include a step determining a likelihood that the dog has a cancer using extrapolation data set. The extrapolating data may be expressed with a linear equation with an R² value higher than 0.9.

The method may involve sending the digital information to an external server via a wireless communication. The digital information may be obtained using a reader box comprising a wireless communication module, a camera module, a light source, and a slot designed to accommodate the lateral flow kit. The wireless communication module operates based on a short range wireless communication protocol instead of a mobile network allowing the method being conducted without any mobile phone but only a short-range communication such as Wi-Fi.

The concentration of the antibody may be determined by the method in a level that is an order of less than about 5 μg or less.

Another embodiment provides a method of determining a concentration of an antibody of ECPKA in blood of a mammal, including (a) preparing a test sample from the blood of the mammal for a lateral flow kit comprising a test line; (b) applying the test sample to the lateral flow kit; (c) allowing the lateral flow kit to develop; (d) obtaining a digital information by taking a digital picture of the developed lateral flow kit including the test line; and (e) obtaining the concentration of the antibody wherein the concentration is determined by comparing the digital information of the test line with a set of data correlating a digital value obtained from the digital picture with a concentration of the antibody, wherein the lateral flow kit includes a first solid phase having an immobilized purified recombinant mammal PKA Cα protein wherein the recombinant mammal PKA Cα protein has an amino acid sequence, a conjugate pad comprising a conjugated coloring agent with an optical density of 5 or higher wherein the conjugated coloring agent is conjugated with a first binding protein, and a second solid phase comprising a second binding protein. The conjugated coloring agent is configured to provide a first color intensity in the first solid phase and a second color intensity in the second solid phase. The first color intensity quantitatively depends on the concentration of the antibody in the blood of the dog and the second color intensity is independent of the concentration of the antibody.

Various embodiments disclosed here may be combined as whole or selectively.

### BRIEF DESCRIPTION OF DRAWINGS

FIG. 1 shows comparison between amino acid sequences of a subunit of human ECPKA and a subunit of canine ECPKA.

FIG. 2 shows comparison between mRNA sequences of a subunit of human ECPKA and a subunit of canine ECPKA.

FIG. 3 illustrates canine ECPKA autoantibody measurements of normal dogs, dogs with benign tumors, dogs with tumors and dogs that have been surgically treated for cancer.

FIG. 4 shows a illustrative receiver operating characteristic graph where A is the area under the ROC curve.

FIG. 5 illustrates an ROC curve of a cancer detection method according to an embodiment of the invention.

FIG. 6 illustrates relationships between CRP level in a plasma and various tested subjects.

FIG. 7 illustrates relationships between CRP level in a plasma and various tested subjects.

FIG. 8 illustrates correlation between CRP measurements and canine ECPKA autoantibodies level.

FIG. 9 illustrates correlations between human ECPKA and canine ECPKA in detecting cancers of dogs.

FIG. 10 illustrates correlations between color intensity obtained using one embodiment lateral flow kit and the concentration of the antibody in a sample wherein the correlation is approximately leaner with R² value higher than 0.9.

FIG. 11 illustrate the top view of a reader according to one embodiment.

FIG. 12 illustrate the bottom plate inside of a reader according to one embodiment.

FIG. 13 illustrate a side view of a reader according to one embodiment.

FIG. 14 illustrate a side view of a reader according to one embodiment.

FIG. 15 illustrate an see-through view of a reader according to one embodiment.

FIG. 16 illustrate an see-through view of a reader according to one embodiment.

FIG. 17 illustrates test results of a kit according to one embodiment.

FIG. 18 illustrates an ROC curve of test results of a kit according to one embodiment.

FIG. 19 summarizes test results of a kit according to one embodiment.

### DETAILED DESCRIPTION OF THE INVENTION

Through extensive researches, it has been found that canine ECPKA protein secrets in a high level and an autoantibody against the canine ECPKA protein is formed in dogs with cancers. It is also found that human ECPKA does not selectively bind to a canine ECPKA autoantibody and cannot serve as a biomarker. In addition, canine ECPKA autoantibody detection can be used as a meaningful diagnosis tool for cancer in dogs only when quantitative measurement of such antibodies is adapted. When the measurement of canine ECPKA autoantibody is not conclusive, measuring CRP can provide supplemental data that can be used to improve the predictability of the canine ECPKA autoantibody measurement as described further below.

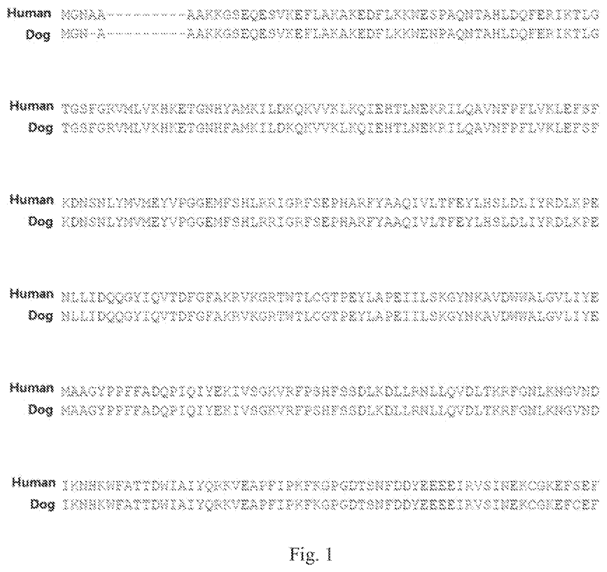

FIG. 1 is alignment of amino acid sequences of PKA Cα from human (NP_002721.1), dog (NP_001003032.1) and cat (XP_006928552.1). Dot boxes indicates amino acid residues showing difference between human and dog. While the amino acid sequences of human and dog share highly similarities but there are four different sequences are spread over the entire sequences including the c-terminus.

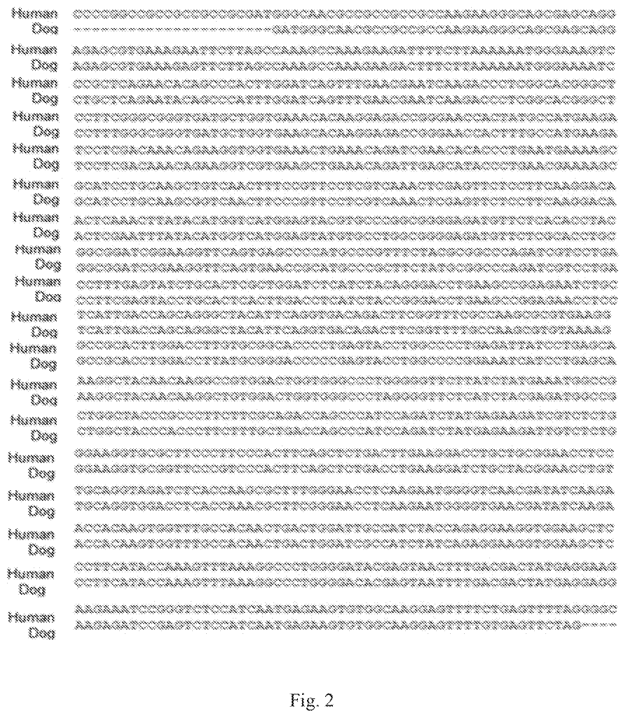

FIG. 2 compares mRNA sequences encoding PKA Cα from human (NP_002721.1), dog (NP_001003032.1) and cat (XP_006928552.1). There are a large number of differences in between mRNA sequences of human and dog.

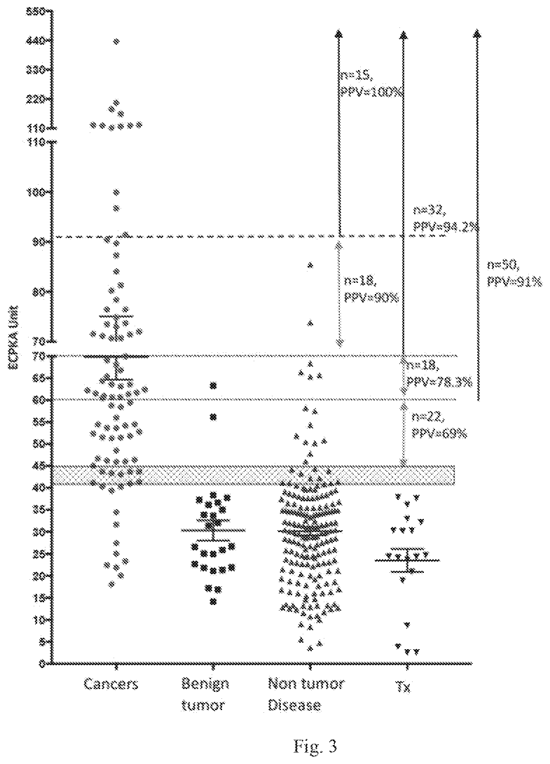

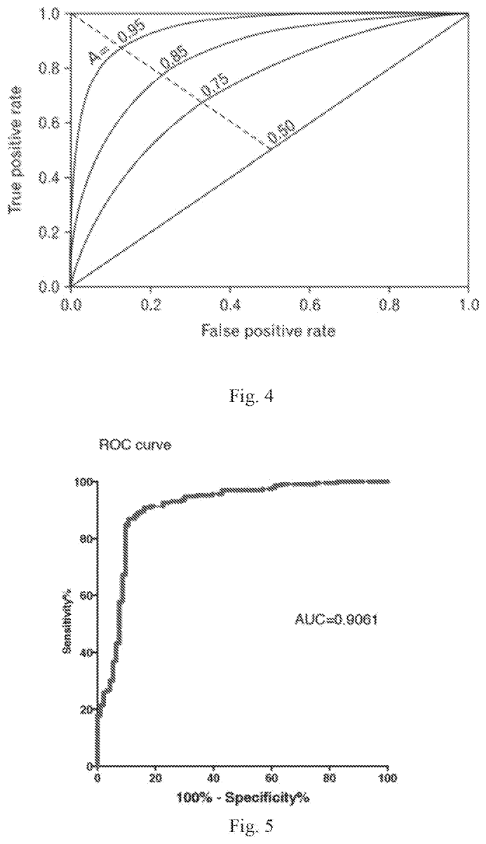

FIG. 3 illustrates the test results of ECPKA autoantibody measurements in various test subjects: dogs (“Cancers” or “Cancer”) diagnosed as having cancer, dogs (“Benign tumor”) with benign tumor, dogs (“Control” or “Non tumor disease”) with no tumor disease, and dogs (“Tx” or “Treatment”). As shown in FIG. 3, the test subjects with cancers show much a higher level of ECPKA autoantibody measurement comparing to the other test subjects. Dogs that had tumor diseases but were treated through surgery show low levels of ECPKA autoantibody measurements. Table 1 summarizes total numbers of the test subjects and test results. For categorizing the test results, the positive results were counted when the ECPKA autoantibody is detected 41 unit and higher, and the negative results were determined when the level of the ECPKA autoantibody detection is less than 41 unit.

TABLE 1

| | Cancer | Control | Benign Tumor | Treatment |
|---|---|---|---|---|
| Positive | 81 | 25 | 2 | 0 |
| Negative | 1 | 160 | 23 | 19 |
| Total | 93 | 185 | 25 | 19 |

Table 2 summarizes the sensitivities, specificity, positive predictive value, and negative predictive value for the canine ECPKA autoantibody detection as a cancer diagnosis method.

TABLE 2

| | | | |
|---|---|---|---|
| Selectivity | 87% | Positive Predictive Value | 75% |
| Specificity | 88.4% | Negative Predictive Value | 94.4% |

There are many ways to verify or evaluate accuracy of a particular test method. Among those, sensitivity and specificity are commonly used. In general, the sensitivity and specificity means how good a method is in distinguishing between the targeted result and untargeted result. For example, in our case, the sensitivity means how well cancers in dogs can be found by using the detection of the autoantibody of canine ECPKA. On the other hand, the specificity relates to how well the method could distinguish dogs with cancer from dogs without cancer. In addition to the sensitivity and specificity, Receiver Operating Characteristics (ROC) curve is often used to determine usefulness and cut-off value of a method. The ROC curve is drawn using the rate of false positive in x axis value and the rate of true positive in y axis value. Whether a particular test method is accurate or not can be measured by using the ROC curve. In the below illustrative ROC curve, in which the positive proportion is plotted against the false positive proportion for various possible settings of the decision criterion, the area under the ROC curve (AUC) of 1 means that the test method is perfect and accurate but if the AUC is 0.5, the test method is useless and inaccurate. As the curve is closer to the upper left corner, the test method is more accurate and more useful. Typically, it is treated that a test method is not informative when the AUC is 0.5, is not so accurate when the AUC is between 0.5 and 0.7, is accurate when the AUC is between 0.7 and 0.9, and is very accurate when the AUC is 0.9 and 1. Accordingly, the ROC curve and its AUC value are a good tool to determine and evaluate a new test method. See Using the Receive Operating Characteristic (ROC) Curve to Measure Sensitivity and Specificity, Korean J. Fam. Med. Vol. 30, No. 11, November 2009, 30:841-842.

FIG. 5 shows a ROC curve of the canine ECKPA autoantibody measurement for detecting cancer of dogs. The AUC value is 0.9061, which indicates that the canine ECKPA autoantibody measurement of one embodiment of the present invention is a highly effective and accurate diagnostic tool.

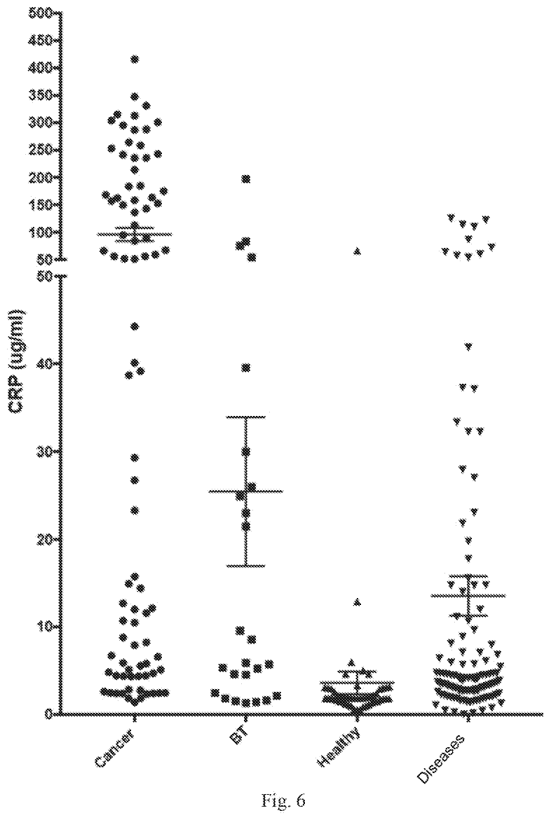

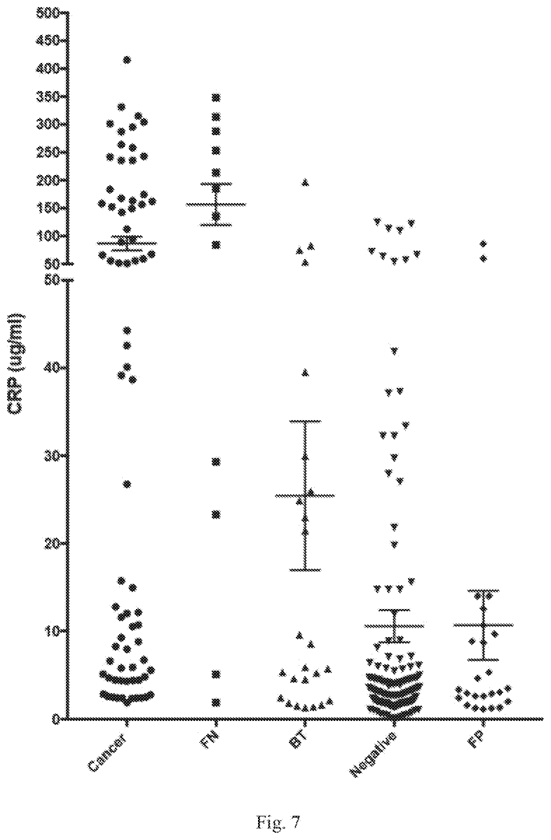

FIG. 6 shows measurements of C-reactive protein (CRP) in various test subjects: dogs with cancer, dogs with benign tumors, dogs with no tumor diseases, dogs with non-cancer diseases. FIG. 7 shows measurements of CRP in dogs with cancer (“Cancer”), dogs with false native results in the canine ECKPA autoantibody measurement (“FN”), dogs with benign tumors (“BT”), dogs without cancer (“Negative”) and dogs with false positive results in the canine ECKPA autoantibody measurement (“FP”).

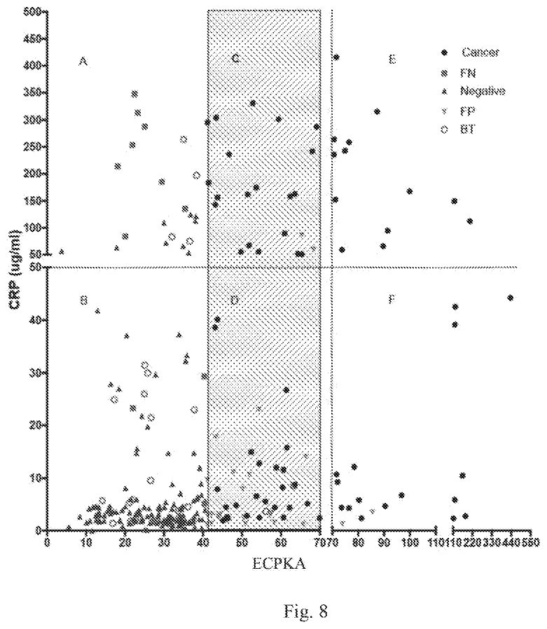

FIG. 8 plots the CRP measurement results against the canine ECKPA autoantibody measurement results of the various test subject groups. Table 3 show negative predictive values and positive predictive values when the CRP and the ECKPA autoantibody measurements are used together. The positive predictive values increase to 91% and 100% from the overall positive predictive value of 73.6% in the areas of C and E, and the negative predictive value improves to 97.6% in the area of C from the overall negative predictive value of 93.4%. Thus, measurement of CRP can improve the accuracy of the cancer diagnosis using the canine ECKPA autoantibody measurement.

TABLE 3

| | | | | Benign | |
|---|---|---|---|---|---|
| Area | NPV (%) | PPV (%) | Cancer | Tumor | Non-tumor |
| A | 62 | 38 | 8 | 4 | 9 |
| B | 97.6 | 2.4 | 4 | 19 | 138 |
| C | 9 | 91 | 20 | 0 | 2 |
| D | 50 | 50 | 25 | 2 | 23 |
| E | 0 | 100 | 13 | 0 | 0 |
| F | 11 | 89 | 16 | 0 | 2 |

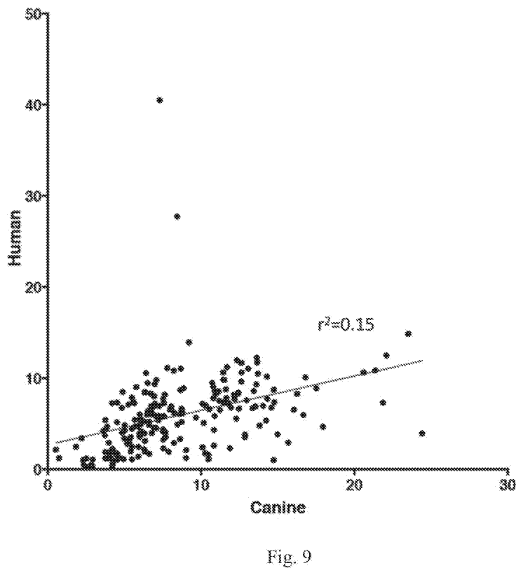

FIG. 9 is a chart that the test results of dogs based on human ECPKA is plotted against the test results of dog based on canine ECPKA. When the r² value is 1, the test results closely correlate to each other and human ECPKA or its autoantibodies can be used to detect cancer in dogs. However, as shown in Fig. x, the r² value is 0.15, suggesting there is not much correlation between human ECPKA and Dog ECPKA and human ECPKA or its autoantibodies is not a good biomaker to detect cancers of dogs.

The table 4 summarizes various cancer detected in the test subjects. It is also found that the cancer detection method of one embodiment of the present invention can be used regardless of the cancer type, which makes the detection method unique and highly useful.

TABLE 4

| Tumor type (Number) | Diagnosis (Number) |
|---|---|
| Carcinoma (57) | Mammary gland Carcinoma (14) |
| | TCC (11) |
| | Hepatic carcinoma (9) |
| | Adenocarcinoma (5) |
| | SCC (5) |
| | Other carcinoma (13) |
| Hematopoietic cancer (2) | Lymphoma (14) |
| | Mast cell tumor (5) |
| | Leukemia (1) |
| Sarcoma (16) | Hemangiosarcoma (5) |
| | Melanoma (4) |
| | Soft tissue sarcoma (4) |
| | Other sarcoma (3) |
| Benign (25) | Adenoma (9) |
| | Lipoma (5) |
| | Histiocytoma (2) |
| | Benign mixed tumor (2) |
| | Other benign tumors (7) |

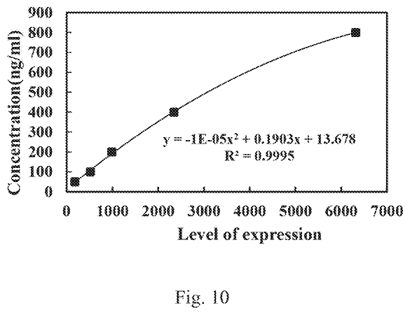

The lateral flow kit structure may follow typical lateral flow kit structures. However, the coloring agent and proteins used are specially designed to detect the antibody of the canine ECPKA in a quantitative way. The kit has an application place where a test sample is applied. The test sample is typically prepared from blood of a test subject. Serum obtained from the blood is applied to the application place. The serum sample flows along the test strip and passes through a conjugate pad where a conjugated coloring agent is placed. The coloring agent is applied with pressure and baked in oven. Typical optical density used in lateral flow kits is around 2-3. However, a much higher optical density is used to obtain better correlation between the digitized test result of the expression and the concentration data. Various coloring agent can be used. Gold and latex are typical coloring agent. For UV active coloring agents include gfp, fitc, and UV active conjugating agents. The color agent exhibits different color intensity. The inherent intensity affects the digitization. FIG. 10 shows an example correlation between the color intensity received after testing using a lateral flow kit according to one embodiment and the concentration of the antibody.

The antibody of the canine ECPKA and other antibodies in the sample will bind to the conjugated first binding protein, effectively coating the antibodies with the coloring agent. The first binding protein may be selected from a group of streptavidin, biotin, protein A, anti-canine IgG Rat, anti-canine IgG Rabbit, anti-canine IgG goat, anti-canine IgG sheep and other protein capable of binding to the antibody. When the sample passes through the first solid phase of the kit, which contains an immobilized purified recombinant canine PKA Cα protein, the antibody of the canine ECPKA binds to the immobilized purified recombinant canine PKA Cα protein. After the complete development of the kit, only the antibody of the canine ECPKA stays in the first solid phase, exhibiting a color intensity, which can be used to find the corresponding concentration of the antibody. The expressed test result is digitized by take a digital camera and the digitized information is compared with the correlation data to determine the actual concentration. The digitized information uses color intensity to obtain a digital expression value. The embodiment shown in FIG. 10 provides a linear relationship between the digitized expression value and the concentration. Thus, it allows extrapolation of a concentration for which matching data does not exist in the data set. For easier extrapolation, the color intensity of the coloring agent for various concentrations of the antibody is desirable to provide a linear relationship.

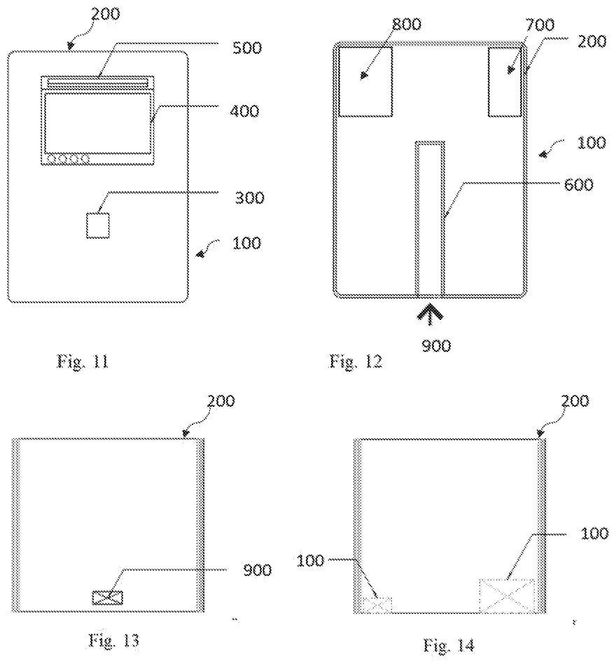

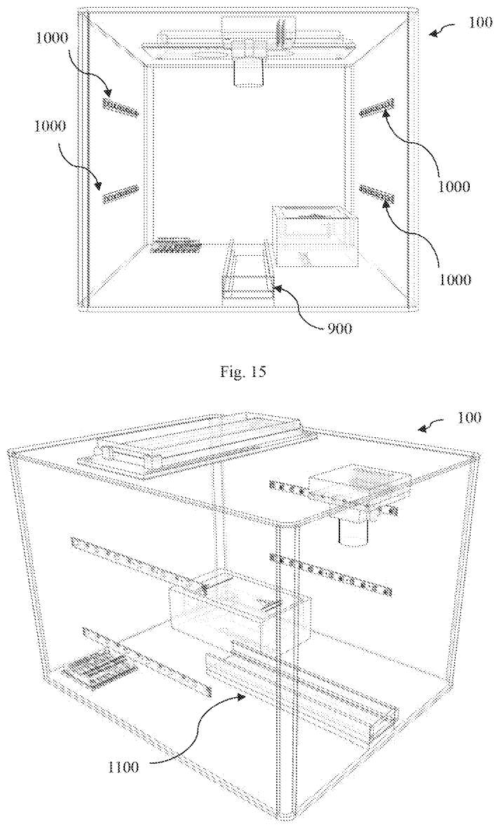

Types of camera, color temperature setting, distance between the sample and camera, light source and exposure setting would affect the digitized value of the color intensity. Thus, it is desirable to have the digitization of the expressed color intensity is desirably done in a constant condition. FIGS. 11-16 shows a reader box 100, which has a housing 200. A camera module 300, monitoring and controlling unit 400 and bar code scanner 500 are provided on the top surface of the housing. Inside the reader, there is a slot 600 where the kit is placed through the opening 900. With the internal lighting system 1000 such as LED, the lighting inside of the reader is controlled to optimize to provide a constant condition for the digitization. When the digital camera module takes a picture of the kit on the slot, the digital image is transferred to a server via the wireless communication module 100. The wireless communication module uses a short distance communication protocol, allowing the reader to connect to a local internet portal such as WI-FI hot spot. The wavelength of the lighting source can be adjusted for various coloring agents such as UV active coloring agents. Because the reader has own communication module, it can operate independent of a mobile network.

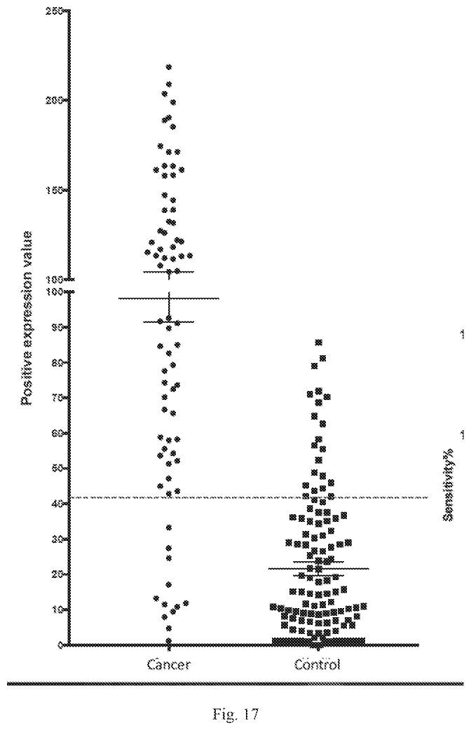

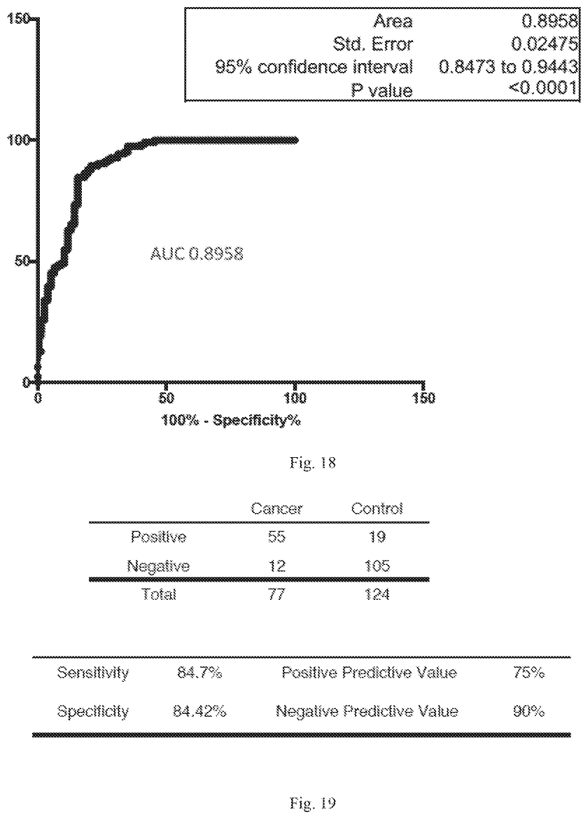

FIGS. 17-19 shows test results of a kit according to an embodiment where the kit shows sensitivity of 84.7% and specificity of 84.4%.

### EXAMPLES

### Cloning of Canine Cyclic AMP-Dependent Protein Kinase Catalytic Subunit Cα (PKA Cα)

Total RNAs were isolated from canine adipose tissue homogenized in Trizol reagent (Invitrogen) using RNeasy columns (Qiagen). 1 μg of total RNA was then reverse transcribed to cDNA with oligo (dT) primers using Improm-II™ Reverse Transcription System (Promega) according to manufacturer's instructions. The canine PKA Cα cDNA was amplified by polymerase chain reaction (PCR) using exTaq polymerase (Takara) and the canine PKA Cα primers containing restriction enzyme recognition sites, NcoI and XhoI. The sequences of primers are as follows: forward, AAT CCA TGG GCA ACG CCG CCG CCA AGA AGG GCA G and reverse, GCC GTC GAC GAA CTC ACA AAA CTC CTT GCC ACA CTT C.

The amplified cDNA fragments of canine PKA Cα was then digested with restriction enzymes, NcoI and XhoI (Takara), inserted into pET-22b(+) plasmid (Novagen), a bacterial expression vector and sequenced with T7 promoter and terminator primers (T7 promoter primer, AAT ACG ACT CAC TAT AGG and T7 terminator primer, GCT AGT TAT TGC TCA GCG G).

### Purification of Recombinant Canine PKA Cα

pET-22b(+) plasmid encoding canine PKA Cα tagged with six histidine residues (6×-His Epitope) at the C-terminus was introduced into Escherichia coli strain, BL21(DE3) and the expression of canine PKA Cα was induced with 1 mM Isopropyl β-D-1-thiogalactopyranoside (IPTG) at room temperature for overnight. Cells were harvested, resuspended in 50 mM Tris.HCl (pH 7.4) containing 0.2M NaCl and sonicated. Recombinant canine PKA Cα was then purified with two sequential immobilized metal affinity chromatography using IDA Excellose resin (Bioprogen) followed by ion exchange chromatography using SP Sepharose resin (GE healthcare). The eluted recombinant protein was dialyzed and stored at a concentration of 1 mg/ml in 50 mM Tris.HCl (pH 7.4) supplemented with 0.15M NaCl and 1% sucrose at −80° C. until further use.

### Western Blotting

50 ng of purified recombinant canine PKA Cα and human PKA Cα, a positive control, proteins were separated on 10% SDS PAGE gel, transferred onto PVDF membrane and sequentially probed with rabbit polyclonal anti-PKA Cα antibody (Abcam) and goat anti-rabbit IgG antibody conjugated with horse radish peroxidase (HRP) (Bethyl Laboratories) followed by immunodetection with enhanced chemiluminescence (Pierce).

### Enzyme-Linked Immunosorbent Assay (ELISA) Assay

The presence and level of extracellular PKA Cα in canine serum was assessed with anti-canine PKA Cα ELISA kit (Genorise) following the manufacturer's instructions. Briefly, 100 μl of 4-fold diluted canine serum samples in reagent diluent was added to the 96 well ELISA plates precoated with anti-canine PKA Cα antibodies and incubated for 1 hr at room temperature. The plates were further incubated with canine PKA Cα detection antibodies for 1 hr at room temperature followed by incubation with HRP conjugate for 20 min at room temperature. The plates were then developed with substrate solution for 10 min at room temperature and the reaction was stopped with 50 μl stop solution. The absorbance was determined at 450 nm with a scanning multi-well spectrophotometer (Molecular Device).

Autoantibodies against extracellular PKA Cα in canine serum were measured using solid phase ELISA method. Briefly, 96 well polystyrene ELISA strip plates (Santa Cruz) were coated with 100 μl of recombinant canine PKA Cα diluted at 1 μg/ml in carbonate coating buffer (pH 9.6) (Sigma) for overnight at room temperature, washed once with PBS containing 0.1% Tween 20 (pH 7.4), blocked with 1% bovine serum albumin (BSA) in PBS for 2 hrs at room temperature and washed twice with washing buffer (50 mM sodium citrate supplemented with 0.15M NaCl and 0.1% Tween 20 (pH 5.2)). The plates were then incubated with 100 μl of canine serum samples diluted at 1:500 in sample dilution buffer (PBS containing 0.25% BSA and 0.05% Tween 20 (pH 7.4)) for 1 hr at room temperature, washed four times with washing buffer, further incubated with 100 μl of goat anti-canine IgG antibody (Abcam) conjugated with HRP diluted at 1:20,000 in sample dilution buffer for 1 hr at room temperature, washed five times with washing buffer, and developed with 100 μl of 3,3′,5,5′-Tetramethylbenzidine (TMB) Liquid Substrate solution (Thermo-Fisher) for 15 min at room temperature. The reaction was then stopped with 50 μl of 2N H₂SO₄ solution and the absorbance was measured at 450 nm using a scanning multi-well spectrophotometer.

Detection of Extracellular PKA Cα in Sera from Dogs with Malignant Tumors

It has been shown that extracellular PKA produced by cancer cells is markedly increased in the sera of cancer patients and elicits the generation of autoantibodies against this it in those patients. In addition, it was reported that the titer of autoantibodies for PKA in serum is significantly correlated with the presence of cancers of various cell types. Hence, the autoantibody against PKA is considered as a novel potential biomarker for diagnosis of cancers in human. However, the presence of PKA autoantibody and its correlation with cancer have never been determined in mammals other than human.

Detection of Autoantibodies against Extracellular PKA Cα in Sera from Dogs with Malignant Tumors

To determine whether the titer of PKA Cα autoantibodies is positively correlated with cancer of various cell types in dogs as shown in humans, first, we cloned canine PKA Cα gene from canine adipose tissues, bacterially expressed and purified the recombinant protein tagged with 6×-His Epitope (Figure). The presence and titer of PKA Cα autoantibodies were then assessed with ELISA assay using the purified canine PKA Cα protein as an antigen.

### Preparation of a Lateral Flow Kit

ECPKA (0.5-4 mg/ml) and control protein (1-2 mg/ml) were diluted protein buffer was dispensed on nitrocellulose membrane by Biodot low volume precision dispensing equipment. The nitrocellulose membrane is dried overnight under 10% humidity. A sample pad is dipped into a sample pad butter and is dried at 37° C. for overnight under 15% humidity. A suspension of high density gold particle conjugated with a protein for the detection of canine IgG is sprayed with pressure into sliced conjugate pad at a room temperature to obtain a high optical density. The conjugation pad was then dried for overnight at 25° C. under 10% humidity. The kit was assembled by putting nitrocellulose membrane on a backing pad, putting the sliced conjugation pad filled with gold particle on backing pad which has to be overlapping with nitrocellulose membrane in front area, putting the sample pad over conjugation pad, putting an adsorption pad overlapping with nitrocellulose membrane in tail part, cutting the assembled backing pad by 4 mm wide and putting the cut assembled backing pad into a housing.

Since the above embodiments are described only for examples, the present invention is not limited to the above embodiments and various modifications or alterations can be easily made therefore by those skilled in the art without departing from the scope of the present invention.

What is claimed is:

1. A lateral flow kit for quantitatively detecting an antibody of canine ECPKA in blood of a dog, comprising:
a first solid phase having an immobilized purified recombinant canine PKA Cα protein wherein the recombinant canine PKA Cα protein has an amino acid sequence comprising SEQ ID NO. 005,
a conjugate pad comprising a conjugated coloring agent with an optical density wherein the conjugated coloring agent is conjugated with a first binding protein, and
a second solid phase comprising a second binding protein,
wherein the conjugated coloring agent is configured to provide a first color intensity in the first solid phase and a second color intensity in the second solid phase, and
wherein the first color intensity quantitatively depends on the concentration of the antibody in the blood of the dog and the second color intensity is independent of the concentration of the antibody

2. The lateral flow kit according to claim 1, wherein the optical density of the conjugated coloring agent is between 5-30.

3. The lateral flow kit according to claim 1, wherein the optical density of the conjugated coloring agent is between 10-20.

4. The lateral flow kit according to claim 1, wherein the lateral flow kit detects cancer in the dog with a specificity of at least about 80% and a sensitivity of at least about 80%.

5. The lateral flow kit according to claim 1, wherein the first color intensity provides the concentration of the antibody by comparing the first color intensity with a set of correlating data between the first color intensity and the concentration of the antibody.

6. The lateral flow kit according claim 5, wherein a portion of the set of correlating data can be expressed approximately by a linear equation, which allows determination of the concentration when the first color intensity does not exactly match with any of the set of correlating data.

7. The lateral flow kit according claim 1, wherein the first binding protein is selected from a group of streptavidin, biotin, protein A, anti-canine IgG Rat, anti-canine IgG Rabbit, anti-canine IgG goat, anti-canine IgG sheep and a protein capable of binding to the antibody.

8. The lateral flow kit according to claim 1, wherein the second binding protein is selected from a group of streptavidin, biotin, protein A, anti-rat IgG, anti-Rabbit IgG, anti-goat IgG, anti-sheep IgG and a protein capable of binding to IgG.

9. The lateral flow kit according to claim 1, wherein the coloring agent is selected from a group of gold, latex, gfp, fitc, and a UV active conjugating agent.

10. The lateral flow kit according to claim 1, further comprising a filter phase to filter blood cells.

11. A method of determining a concentration of an antibody of canine ECPKA in blood of a dog, comprising:
(a) preparing a test sample from the blood of the dog for a lateral flow kit,
(b) applying the test sample to the lateral flow kit,
(c) allowing the lateral flow kit to develop,
(d) obtaining a digital information by taking a digital picture of the developed lateral flow kit including the test line, and
(e) obtaining the concentration of the antibody wherein the concentration is determined by comparing the digital information with a set of data correlating a digital value with a concentration of the antibody
wherein the lateral flow kit comprises
a first solid phase having an immobilized purified recombinant canine PKA Cα protein wherein the recombinant canine PKA Cα protein has an amino acid sequence comprising SEQ ID NO. 5,
a conjugate pad comprising a conjugated coloring agent with an optical density wherein the conjugated coloring agent is conjugated with a first binding protein, and
a second solid phase comprising a second binding protein,
wherein the conjugated coloring agent is configured to provide a first color intensity in the first solid phase and a second color intensity in the second solid phase
wherein the first color intensity quantitatively depends on the concentration of the antibody in the blood of the dog and the second color intensity is independent of the concentration of the antibody

12. The method according to claim 11, further comprising determining a likelihood that the dog has a cancer.

13. The method according to claim 11, further comprising sending the digital information is sent to an external server via a wireless communication.

14. The method according to claim 11, wherein the obtaining the digital information is conducted using a reader box comprising a wireless communication module, a camera module, a light source, and a slot designed to accommodate the lateral flow kit.

15. The method according to claim 14 wherein the wireless communication module operates based on a short range wireless communication protocol.

16. The method according to claim 11, wherein the optical density is higher than 5.

17. The method according to claim 11, wherein the optical density is higher than 10.

18. The method according to claim 11 wherein the method detects cancer in the dog with a specificity of at least about 80% and a sensitivity of at least about 80%.

19. The method according to claim 11, wherein the concentration of the antibody is extrapolated using a linear equation with an R² value higher than 0.9.

20. A method of determining a concentration of an antibody of ECPKA in blood of a mammal, comprising:
(a) preparing a test sample from the blood of the mammal for a lateral flow kit comprising;
(b) applying the test sample to the lateral flow kit;
(c) allowing the lateral flow kit to develop;
(d) obtaining a digital information by taking a digital picture of the developed lateral flow kit; and
(e) obtaining the concentration of the antibody wherein the concentration is determined by comparing the digital information of the test line with a set of data correlating a digital value obtained from the digital picture with a concentration of the antibody
wherein the lateral flow kit comprises
a first solid phase having an immobilized purified recombinant mammal PKA Cα protein wherein the recombinant mammal PKA Cα protein has an amino acid sequence,
a conjugate pad comprising a conjugated coloring agent with an optical density of 5 or higher wherein the conjugated coloring agent is conjugated with a first binding protein, and
a second solid phase comprising a second binding protein,
wherein the conjugated coloring agent is configured to provide a first color intensity in the first solid phase and a second color intensity in the second solid phase, and
wherein the first color intensity quantitatively depends on the concentration of the antibody in the blood of the dog and the second color intensity is independent of the concentration of the antibody.
